(19) United States
(12) Patent Application Publication
(10) Pub. No.: US 2019/0234933 A1
(43) Pub. Date: Aug. 1, 2019

(54) COMPOSITIONS AND ASSAYS

(71) Applicant: GENADAM THERAPEUTICS, INC., Jamaica Plain, MA (US)

(72) Inventor: David SHERRIS, Jamaica Plain, MA (US)

(21) Appl. No.: 16/265,705

(22) Filed: Feb. 1, 2019

Related U.S. Application Data
(60) Provisional application No. 62/625,138, filed on Feb. 1, 2018.

(51) Int. Cl. G01N 33/50

(52) U.S. Cl. CPC G01N 33/502; G01N 2500/10

(57) ABSTRACT

The present disclosure contemplates methods for identifying candidate compounds or gene products useful in treating or ameliorating bacterial, fungal or viral infections; or for treating or ameliorating cancers or tumors characterized by interruption of pyroptosis. Compositions, methods of making, as well as kits, which utilize such pharmaceutical composition are also contemplated.

### FIELD OF THE DISCLOSURE

The fields of the disclosure include at least immunology, virology, cell biology, molecular biology, and medicine, including anti-bacterial, anti-fungal, anti-viral and cancer medicine.

### BACKGROUND

Pyroptosis is a highly inflammatory form of programmed cell death that occurs most frequently upon infection with intracellular pathogens and forms part of the immune system's antimicrobial response. In this process, immune cells recognize foreign danger signals within themselves after ingesting a pathogen, which causes the immune cells to release pro-inflammatory cytokines capable of bringing in and activating innate and adaptive immune cells, swell, burst and die. The term pyroptosis is coined from the Greek word pyro, meaning fire or fever, and ptosis, meaning falling, which reflects its inflammatory nature and highlights that it is distinct from other forms of programmed cell death. Pyroptosis requires the function of the enzyme caspase-1, which is activated by an inflammasome.

Generally, an inflammasome is a multiprotein complex that initiates the maturation and release of the cytokines IL-1β and IL-18 via the activation of caspase-1. When cytosolic pattern recognition receptors within macrophage or dendritic cells sense either pathogen associated molecular patterns or danger associated molecular patterns, an active inflammasome is assembled along with the generation of activated caspase-1. Inflammasomes have been shown to be formed in macrophage or dendritic cells as soon as minutes after ingestion of a pathogen.

Inflammasomes activate caspase-1 in response to cytosolic contamination or perturbation. Caspase-1 is an inflammatory enzyme, and triggers the opening of the gasdermin-D (GSDMD) pore in the plasma membrane, resulting in pyroptotic cell death. Viable bacteria remain trapped within the cellular debris of pyroptotic macrophage or dendritic cells. Although membrane tears release soluble cytosolic contents, they are small enough to retain organelles and bacteria. This structure has been called “the pore-induced intracellular trap” (PIT). The PIT coordinates innate immune responses via complement and scavenger receptors to drive recruitment of and efferocytosis by neutrophils. Ultimately, a secondary macrophage, dendritic cell, or neutrophil, is called to kill the still-viable bacteria. Hence, caspase-1—driven pore-induced cell death triggers a multifaceted defense against intracellular bacteria facilitated by trapping the pathogen within the cellular debris.

Many pathogens replicate within this macrophage or dendritic cell intracellular niche. If inflammasomes succeed in detecting the pathogen, pyroptosis effectively eliminates the protected intracellular niche. After cell death, the organelles, cytoskeleton, and intracellular bacteria remain within the ruptured but largely intact plasma membrane. The bacteria entrapped in the debris are not killed, and are only killed after phagocytosis of the PIT and entrapped bacterium by a neutrophil. However, some intracellular bacterial pathogens, such as Salmonella, Shigella, Francisella, Listeria or tuberculosis (i.e., Mycobacterium tuberculosis), possess adaptations to prevent or delay pyroptosis and subsequently killed by neutrophils.

Some pathogens have evolved mechanisms to inhibit pyroptosis, enhancing their ability to persist and cause disease. Ultimately, there is a competition between host and pathogen to regulate pyroptosis, and the outcome dictates life or death of the host. For example, Legionella and Salmonella, both being bacterial species having flagella, use translocation systems to modulate host cell function, but must also avoid introducing flagellin into the cytosol through these translocation systems and stimulating pyroptosis, since flagellin is recognized by inflammasomes. Both organisms downregulate flagellin production during intracellular growth, which could provide a strategy to avoid pyroptosis, thereby limiting inflammation and allowing continued intracellular replication of the bacteria.

Other pathogens escape pyroptosis by inducing alternative forms of cell death, such as apoptosis. Yersinia can trigger apoptosis in macrophage and dendritic cells, which effectively prevents inflammatory pyroptosis. Pseudomonas strains that produce the type III secretion system-secreted protein ExoU induce caspase 1-independent necrosis, resulting in lysis but preventing the cleavage and release of IL-1β and IL-18.

Some pathogens also produce factors that can directly inhibit the activation of caspase-1. For example, the poxvirus protein M13L-PYD binds ASC through its pyrin domain, thereby disrupting inflammasome formation and preventing activation of caspase-1. The influenza virus protein NS1 has also been shown to limit caspase-1 activation and cell death through a mechanism still not well understood. This indicates that inhibition of caspase-1 activation could be a common strategy for successful viral pathogens. Yersinia translocates type III secretion proteins that counteract the caspase-1 activating potential of the type III secretion system itself. Francisella mutants that trigger induction of pyroptosis more quickly than the wild type have been identified, suggesting that Francisella also possesses a mechanism for inhibiting caspase-1 (See Weiss D S, et al., “In vivo negative selection screen identifies genes required for Francisella virulence,” Proc Natl Acad Sci USA. 2007 Apr. 3; 104(14):6037-42), and Mycobacterium tuberculosis produces a zinc metalloprotease that prevents activation of caspase-1 through an unknown mechanism (Master SS, et al., “Mycobacterium tuberculosis prevents inflammasome activation” Cell Host Microbe. 2008 Apr. 17; 3(4):224-32).

In addition, it is believed that several types of cancers and tumors may be treatable by inducing the activation and release of caspase enzymes, such as caspase-1. For example, schwannomas in peripheral distal and intracranial nerves are the hallmark of neurofibromatosis 1 and 2 (NF1 and NF2), and schwannomatosis, three types of nerve sheath tumors. The underlying molecular abnormality in NF2 is a germline mutation of the NF2 gene. Somatic loss of the normal remaining NF2 allele in Schwann cells leads to deregulated growth of neoplastic Schwann cells with schwannoma formation. Similar mutations in NF1 and schwannomatosis occur in NF1 and NF2, respectively. One particular gene target has been the caspase family. Of the caspases family of genes, caspases-3 is the most common target for therapeutic modulation. Unlike caspase-3, caspase-1 has a strong pro-inflammatory component in addition to induction of apoptosis. Caspase-1 activates IL-1β and IL-18, which in turn trigger immune responses mediated by neutrophils and monocytes, or NK cells. In addition, caspases-1 has been associated with both innate and adaptive immunity and is activated by several chemotherapeutic drugs which can sensitize tumors cells to chemotherapy and radiation.

A hallmark of cancer is that tumor cells escape from or are resistant to programmed cell death mediated by apoptosis or programmed necrosis or by being ignited by other lethal cascades, including mitotic catastrophe. Inflammasome dependent cell death, pyroptosis, is thought to be dysregulated during oncogenesis and tumor progression. For example, caspase-1 is downregulated in human prostate cancers, and reintroduction of CASP1 in prostate cancer cells leads to greater sensitivity to radiation-induced killing in vitro. Winter, R N, et al., Loss of caspase-1 and caspase-3 protein expression in human prostate cancer. Cancer Res. 61,1227-1232 (2001); Winter, R N, et al., “Caspase-1 enhances the apoptotic response of prostate cancer cells to ionizing radiation.” Anticancer Res. 24, 1377-1386 (2004). NLRP3−/−(NLRP3 inflammasome) mice are more susceptible to colitis-associated colon cancer in the azoxymethane-dextran sulfate sodium (AOM-DSS) model than wild-type mice. Allen, I C et al. The NLRP3 inflammasome functions as a negative regulator of tumorigenesis during colitis-associated cancer. J. Exp. Med. 207, 1045-1056 (2010). Furthermore, IL-1β activation through caspase-1 is known to have an essential role in stimulating adaptive immune responses resulting in anticancer immunosurveillance. Eisenbarth, S C & Flavell, R A, Innate instruction of adaptive immunity revisited: the inflammasome. EMBO Mol. Med. 1, 92-98 (2009). By binding to IL-1R1, IL-1β attracts γδ T cells to the tumor site, which leads to local production of IL-17, the recruitment of IFN-γ-producing CD8+ αβ T cells and lysis of tumor cells. Moreover, anthracyclin-based adjuvant chemotherapy is relatively inefficient in patients with breast cancer who have a loss-of-function allele of P2RX7 that negatively affects the production of pyroptotic synthesis and activation of IL-1β and IL-18 via inflammasome activated capase-1. Sluyter, R, et al., P2X7 receptor polymorphism impairs extracellular adenosine 5′-triphosphate-induced interleukin-18 release from human monocytes. Genes Immun. 5, 588-591 (2004).

Thus, there is a need to develop compounds and pharmaceutical compositions that are capable of activating the inflammatory response that leads to pyroptosis to aid the immune system against bacteria, fungus, viral infections or cancers that inhibit pyroptotic cellular response. No assays or methods of identification have been proposed in the prior art. Development of such assays or methods of identification could provide a boost to research in treatments for pathogens that interrupt pyroptosis.

### BRIEF SUMMARY

In one aspect, the current disclosure provides assays and methods for identifying candidate compounds that could be used in a treatment against certain bacterial, fungal or viral infections. Once identified, these compounds may be used in treatments to induce pyroptosis, which generates a therapeutic cascade that leads to induction of host immune responses capable of killing pathogenic organisms that evade immune response by inhibiting pyroptosis. Pyroptosis refers to a biological intracellular cascade that results in cell death and release of biological mediators that induce host immune responses, both innate and adaptive. These secondary host immune responses have the capacity to kill remaining local and distant tumor cells and to establish a vaccine effect. Thus, in some embodiments, the present disclosure is directed to a method of establishing an immunity to certain pathogens.

In another aspect, the current disclosure provides assays and methods for identifying candidate compounds that could be used in method of treating tumors. Once identified, these compounds may be used in treatments to induce pyroptosis, which generates a therapeutic cascade that leads to tumor cell killing and induction of host immune responses capable of killing distal and subsequently arising tumors. In some embodiments, the identified compounds may be used in combination with traditional cancer medications.

### DETAILED DESCRIPTION

In a first aspect, the disclosure is directed to a method (Method 1) for identifying candidate compounds or gene products useful in treating or ameliorating bacterial, fungal or viral infections comprising the steps of:
a. providing a first sample and a second sample, e.g., a bacterial culture or cell or tissue sample, both infected with a bacteria, fungus or virus, said first and second samples containing equivalent amounts of a macrophage or dendritic cell; and optionally
b. contacting the first sample with one of the candidate compounds or gene products;
c. and measuring and comparing the amounts of inflammatory biomarkers in the first sample with the second sample, wherein an increased amount of the inflammatory biomarker in comparison with the second sample indicates that the candidate compound can be useful to treat or ameliorate bacterial or viral infections characterized by interrupted pyroptosis.

For example, Method 1 can further encompass:
1.1 Method 1, further comprising the steps of:
   d. providing a third sample and a fourth sample e.g., a bacterial culture or cell or tissue sample, both free of infection from the bacteria, fungus or virus, said third and fourth samples containing equivalent amounts of a macrophage or dendritic cell;
   e. contacting the third sample with one of the candidate compounds or gene products; and
   f. measuring and comparing the amounts of inflammatory biomarkers in the first sample with the third sample and/or fourth sample, wherein an increased amount of the inflammatory biomarker in comparison with the second sample indicates that the candidate compound can be useful to treat or ameliorate bacterial or viral infections characterized by interrupted pyroptosis.
1.2 Method 1 or 1.1, wherein the inflammatory biomarker is an inflammatory cytokine or a caspase enzyme.
1.3 Any preceding Method, wherein the inflammatory biomarker is a caspase, a pro-caspase, IL-β or IL-18, or combinations thereof.
1.4 Any preceding Method, wherein the inflammatory biomarker is caspase-1, caspase-3, caspase-4, caspase-5, caspase-11, caspase-12, IL-β or IL-18, or combinations thereof.
1.5 Any preceding Method, wherein the inflammatory biomarker is caspase-1, caspase-3 caspase-4, caspase-5, caspase-11, caspase-12 or combinations thereof.
1.6 Method 1 or 1.1-1.2, wherein the inflammatory biomarker is a pro-caspase (e.g, pro-caspase-1).
1.7 Any preceding Method, wherein the inflammatory biomarker is human caspase-1.
1.8 Any preceding Method, wherein the inflammatory biomarker is human pro-caspase-1.
1.9 Any preceding Method, wherein the first and second samples are infected with the same bacterial, fungal or viral infection.
1.10 Any preceding Method, wherein the first and second samples are infected with the same bacterial infection.
1.11 Any preceding Method, wherein the bacterial infection is a Salmonella, Shigella, Francisella, Listeria, Mycobacterium species or strain (i.e., Mycobacterium tuberculosis), Acinetobacter baumannii, Pseudomonas aeruginosa, Enterobacteriaceae, Enterococcus faecium, Staphylococcus aureus, Helicobacter pylori, Campylobacter, Neisseria gonorrhoeae, Streptococcus pneumoniae, Haemophilus influenzae infection.
1.12 Any preceding Method, wherein the bacterial infection is a Mycobacterium tuberculosis infection.
1.13 Any preceding Method, wherein the macrophage or dendritic cell is an M1 or M2 macrophage.
1.14 Any preceding Method, wherein the macrophage or dendritic cell is an M1 macrophage.
1.15 Any preceding Method, wherein a successful candidate compound results in effective treatment against the infecting bacteria, fungus or virus.
1.16 Any preceding Method, wherein a successful candidate is marked by a 50% reduction in bacterial, fungal or viral viability, a 60% reduction in bacterial or viral viability, a 70% reduction in bacterial or viral viability, an 80% reduction in bacterial or viral viability, a 90% reduction in bacterial or viral viability, a 95% reduction in bacterial or viral viability, or greater.
1.17 Method 1, wherein the gene product is a caspase gene or variant thereof selected from the group consisting of: caspase 1, caspase-4, caspase-3, caspase-5, caspase-11, and caspase-12.
1.18 Method 1.17, wherein the caspase gene or variant thereof are a pro-caspase gene (e.g, pro-caspase-1).
1.19 Methods 1.17-1.18, wherein the caspase gene or variant thereof is caspase-1.
1.20 Methods 1.17-1.19, wherein the caspase gene or variant thereof is a human, mouse, or rat caspase (e.g., human caspase-1, human pro-caspase 1).
1.21 Methods 1.17-1.20, wherein the gene product is delivered by viral vector.
1.22 Methods 1.17-1.21, wherein the viral vector comprises a heterologous gene/genes.
1.23 Method 1.22, wherein the heterologous gene/genes are selected from: genes not found in the viral genome; any allelic variant of a wild-type gene; and mutant gene(s).
1.24 Method 1.23, wherein Heterologous genes are preferably operably linked to a control sequence permitting expression of said heterologous gene in a cell in vivo.
1.25 Methods 1.21-1.24, wherein the viral vector can deliver a heterologous gene/genes to a cell in vivo where it will be expressed.
1.26 Methods 1.17-1.25, wherein the genes encode proteins which are cytostatic, cytotoxic, pro-drug activating, or which may be capable of stimulating/enhancing an immune response.
1.27 Methods 1.17-1.26, wherein the heterologous gene/genes are delivered by a single virus.
1.28 Methods 1.17-1.27, wherein the gene product comprises a viral vector comprising a pro-caspase gene (e.g., human pro-caspase-1) or a variant thereof and a promoter.
1.29 Methods 1.17-1.28, wherein the gene product further comprises an additional gene (e.g., human Caspase-4, human Caspase-3, human Caspase-5, mouse Caspase-11, mouse Caspase-12, NLRP1, Nlrp3, NRLC4, NALP, AIM2, PYCARD-derived adaptor proteins CARD or ASC, IL-β and IL-18).
1.30 Methods 1.17-1.29, wherein the gene product encodes a human pro-caspase-1 or a human caspase-1.
1.31 Methods 1.17-1.30, wherein the gene product is delivered by viral vector and wherein the viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, Adeno-associated Virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
1.32 Methods 1.17-1.31, wherein the gene product is delivered by viral vector and wherein the viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, Adeno-associated, Virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
1.33 Any of the preceding Methods, wherein the first sample and the second sample are taken from a patient to be treated for a bacterial, fungal or viral infection.

In various embodiments, the present disclosure also provides for a composition (Composition 1) comprising a compound or gene product identified in Method 1, et seq.

For example, Composition 1 may further encompass:
1.1 Composition 1, wherein the identified compound or gene product induces pyroptosis in macrophage or dendritic cells in the immune system.
1.2 Any preceding Composition, wherein the identified compound or gene product activates and causes release of a caspase, a pro-caspase, IL-β or IL-18, or combinations thereof.
1.3 Any preceding Composition, wherein the identified compound or gene product activates and causes release of caspase-1, caspase-3, caspase-4, caspase-5, caspase-11, caspase-12, IL-β or IL-18, or combinations thereof.
1.4 Any preceding Composition, wherein the identified compound or gene product activates and causes release of caspase-1, caspase-3, caspase-4, caspase-5, caspase-11, caspase-12 or combinations thereof.
1.5 Any preceding Composition, wherein the identified compound or gene product activates and causes release of a pro-caspase (e.g, pro-caspase-1).
1.6 Any preceding Composition, wherein the identified compound or gene product activates and causes release of caspase-1.
1.7 Any preceding Composition, wherein the identified compound or gene product causes release of pro-caspase-1.
1.8 Any preceding Composition, wherein the identified compound or gene product is administered in combination with an antibacterial drug.
1.9 Any preceding Composition, wherein the identified compound or gene product is administered in combination with isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin, capreomycin, ethionamide, cycloserine, para-aminosalicylic acid, thiacetazone, clarithromycin, amoxicillin-clavulanic acid, imipenem, meropenem, clofazimine, viomycin, terizidone, TMC207, PA-824, OPC-7683, LL-3858 and SQ-109.
1.10 Any preceding Composition, wherein the gene product is a caspase gene or variant thereof selected from the group consisting of: caspase 1, caspase-3, caspase-4, caspase-5, caspase-11, and caspase-12.
1.11 Any preceding Composition, wherein the caspase gene or variant thereof are a pro-caspase gene (e.g, pro-caspase-1).
1.12 Any preceding Composition, wherein the caspase gene or variant thereof is caspase-1.
1.13 Any preceding Composition, wherein the caspase gene or variant thereof is a human, mouse, or rat caspase (e.g., human caspase-1, human pro-caspase 1)
1.14 Any preceding Composition, further comprising a viral vector to deliver the gene product.
1.15 Composition 1.14, wherein the viral vector comprises a heterologous gene/genes.
1.16 Composition 1.15, wherein the heterologous gene/genes are selected from: genes not found in the viral genome; any allelic variant of a wild-type gene; and mutant gene(s).
1.17 Composition 1.15-1.16, wherein Heterologous genes are preferably operably linked to a control sequence permitting expression of said heterologous gene in a cell in vivo.
1.18 Composition 1.15-1.17, wherein the viral vector can deliver a heterologous gene/genes to a cell in vivo where it will be expressed.
1.19 Composition 1.15-1.18, wherein the genes encode proteins which are cytostatic, cytotoxic, pro-drug activating, or which may be capable of stimulating/enhancing an immune response.
1.20 Composition 1.15-1.19, wherein the heterologous gene/genes are delivered by a single virus.
1.21 Any preceding Composition, wherein the gene product comprises a viral vector comprising a pro-caspase gene (e.g., human pro-caspase-1) or a variant thereof and a promoter.
1.22 Any preceding Composition, wherein the gene product further comprises an additional gene (e.g., human Caspase-3, human Caspase-4, human Caspase-5, human Caspase-11, mouse Caspase-11, mouse Caspase-12, NLRP1, Nlrp3, NRLC4, NALP, AIM2, PYCARD-derived adaptor proteins CARD or ASC, IL-1β and IL-18).
1.23 Methods 1.16-1.28, wherein the gene product encodes a human pro-caspase-1 or a human caspase-1.
1.24 Any preceding Composition, wherein the gene product is delivered by viral vector and wherein the viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, Adeno-associated Virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
1.25 Any preceding Composition, wherein the gene product is delivered by viral vector and wherein the viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
1.26 Any of the preceding Methods, wherein the first sample and the second sample are taken from a patient to be treated for a bacterial, fungal or viral infection

The compounds or gene products identified via Method 1, et seq., may be used in pharmaceutical compositions to treat a bacterial, fungal or viral infection.

In some embodiments, the pharmaceutical compositions are administered in combination with one or more antibacterial drugs, for example, drugs known to have an effect in treating or eliminating bacterial infections resulting from Salmonella, Shigella, Francisella, Listeria or Mycobacterium tuberculosis, Acinetobacter baumannii, Pseudomonas aeruginosa, Enterobacteriaceae, Enterococcus faecium, Staphylococcus aureus, Helicobacter pylori, Campylobacter, Neisseria gonorrhoeae, Streptococcus pneumoniae, Haemophilus influenzae infection. Non-limiting examples of antibacterial drugs are: (R)-iclaprim, (R)-temafloxacin, (S)-iclaprim, (S)-temafloxacin, 4-{((R)-2-Carboxy-3-methoxymethyl-8-oxo-5-thia-1-aza-bicyclo [4.2.0] oct-2-en-7-ylcarbamoyl)-[(Z)-methoxyimino]-methyl}-thiazol-2-yl-ammonium, amikacin, amikacin disulfate, amoxicillin, amoxicillin trihydrate, ampicillin, apramycin, arasertaconazole, arbekacin, Augmentin, avibactam, avibactam sodium, Avycaz, azithromycin, azlocillin, aztreonam, benzyl isothiocyanate, benzylpenicillin, biapenem, brodimoprim, carbapenem MM22383, carbenicillin, carumonam, cefaclor, cefadroxil, cefaloridine, cefalotin, cefamandole, cefamandole nafate, cefamandole sodium, cefatrizine, cefazedone, cefazolin, cefcapene, cefdinir, cefditoren, cefditoren pivoxil, cefepime, cefepime hydrochloride, cefixime, cefmenoxime, cefmetazole, cefodizime, cefonicid, cefoperazone, ceforanide, cefotaxime, cefotetan, cefotetan disodium, cefotiam, cefotiam dihydrochloride, cefotiam hexetil dihydrochloride, cefoxitin, cefpiramide, cefpodoxime, cefpodoxime proxetil, cefprozil, cefsulodin, ceftaroline, ceftaroline fosamil, ceftaroline, fosamil acetate, ceftaroline fosamil acetate monohydrate, ceftazidime, ceftibuten, ceftibuten dihydrate, ceftizoxime, ceftriaxone, cefuzonam, cephalexin, cephalexin monohydrate, cephapirin, cephapirin sodium, cephradine, chloramphenicol, chlorphenesin, chlorquinaldol, chlortetracycline, cinoxacin, ciprofloxacin, ciprofloxacin dihydrochloride, ciprofloxacin hydrochloride (anhydrous), ciprofloxacin hydrochloride hydrate, clarithromycin, clavulanic acid, clometocillin, clomocycline, cloxacillin, co-trimoxazole, ciclacillin, dalbavancin, daptomycin, demeclocycline, demeclocycline hydrochloride, dicloxacillin, difloxacin, doxycycline, doxycycline hyclate, doxycycline monohydrate, enoxacin, ertapenem, ertapenem sodium, fenticonazole, fenticonazole nitrate, fidaxomicin, finafloxacin, fleroxacin, flomoxef, flucloxacillin, flurithromycin, framycetin, fumagillin, furagin, furaltadone, furazolidone, garenoxacin, Gemifloxacin, hetacillin, hexamethylenetetramine, humulone, iclaprim, imipenem, isoconazole, isoconazole nitrate, josamycin, kanamycin A sulfate, levofloxacin, linezolid, loracarbef, lymecycline, malachite green cation, marbofloxacin, mecillinam, meropenem, meropenem trihydrate, methacycline, methicillin, metronidazole, metronidazole benzoate, metronidazole hydrochloride, mezlocillin, minocycline, moxalactam, moxifloxacin, moxifloxacin hydrochloride, mupirocin, nadifloxacin, nafcillin, nalidixic acid, nifurtoinol, nitrofurantoin, nitrofurazone, norfloxacin, ofloxacin, oritavancin, oritavancin bisphosphate, ornidazole, oxacillin, oxolinic acid, oxytetracycline, paromomycin, paromomycin sulfate, pefloxacin, pefloxacin mesylate, penamecillin, penicillin, pipemidic acid, piperacillin, pivmecillinam, potassium clavulanate, pristinamycin, pristinamycin IA, pristinamycin IIA, prontosil, ribostamycin, ribostamycin sulfate, ristocetin, rolitetracycline, rosoxacin, roxarsone, roxithromycin, salicylhydroxamic acid, sertaconazole, sertaconazole nitrate, silver(1+) sulfadiazinate, sirolimus, spectinomycin, spectinomycin dihydrochloride, spectinomycin hydrochloride hydrate, spiramycin I, spiramycin II, streptomycin, sulfacetamide, sulfachloropyridazine, sulfadoxine, sulfamazone, sulfamethazine, sulfaperin, sulfaphenazole, sulfathiourea, sulfisoxazole, sulfisoxazole diolamine, teicoplanin, telavancin, telavancin hydrochloride, temafloxacin, tetracycline, tetracycline zwitterion, thiostrepton, thiram, ticarcillin, tigecycline, tinidazole, trimethoprim, trovafloxacin, trovafloxacin mesylate, vancomycin, virginiamycin, virginiamycin S1, xibornol, antimycobacterial drug, and antitreponemal drug.

Non-limiting examples of preferred antibacterial drugs are: isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin, capreomycin, ethionamide, cycloserine, para-aminosalicylic acid, thiacetazone, clarithromycin, amoxicillin-clavulanic acid, imipenem, meropenem, clofazimine, viomycin, terizidone, TMC207, PA-824, OPC-7683, LL-3858 and SQ-109.

In a further aspect, the disclosure is directed to a method (Method 2) for identifying candidate compounds useful in treating or ameliorating a cancer or tumor comprising the steps of:
a. providing a first sample and a second sample, e.g., a cancerous cell or tissue sample;
b. contacting the first sample with one of the candidate compounds;
c. and measuring and comparing the amounts of inflammatory biomarkers in the first sample with the second sample, wherein an increased amount of the inflammatory biomarker in comparison with the second sample indicates that the candidate compound can be useful to treat or ameliorate a cancer or tumor characterized by interrupted pyroptosis.

For example, Method 2 can further encompass:
2.1 Method 2, wherein the inflammatory biomarker is an inflammatory cytokine or a caspase enzyme.
2.2 Method 2 or 2.1, wherein the inflammatory biomarker is a caspase, a pro-caspase, IL-β or IL-18, or combinations thereof.
2.3 Any preceding Method, wherein the inflammatory biomarker is caspase-1, caspase-3, caspase-4, caspase-5, caspase-11, caspase-12, IL-β or IL-18, or combinations thereof.
2.4 Any preceding Method, wherein the inflammatory biomarker is caspase-1, caspase-3, caspase-4, caspase-5, caspase-11, caspase-12 or combinations thereof.
2.5 Method 2 or 2.1-2.2, wherein the inflammatory biomarker is a pro-caspase (e.g, pro-caspase-1).
2.6 Any preceding Method, wherein the inflammatory biomarker is human caspase-1.
2.7 Any preceding Method, wherein the inflammatory biomarker is human pro-caspase-1.
2.8 Any preceding Method, wherein the first and second samples are of the same type of cancerous cell or tissue.
2.9 Any preceding Method, wherein the first and second sample originates from a single source.
2.10 Any preceding Method, wherein the compound is useful in treating tumors.
2.11 Any preceding Method, wherein the tumor is benign, pre-malignant or malignant.
2.12 Any preceding Method, wherein the tumor is selected from one or more of acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), schwannoma, adenomas (e.g., basophilic adenoma, eosinophilic adenoma, chromophobe adenoma, parathyroid adenoma, islet adenoma, fibroadenoma), fibroids (fibrous histiocytoma), fibromas, hemangiomas, lipomas (e.g., angiolipoma, myelolipoma, fibrolipoma, spindle cell lipoma, hibernoma, atypical lipoma), myxoma, osteoma, preleukemias, rhadomyoma, papilloma, seborrheic keratosis, skin adnexal tumors, hepatic adenomas, renal tubular adenoma, bile duct adenoma, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, nevus, actinic keratosis, cervical dysplasia, metaplasia (e.g., metaplasia of the lung), leukoplakia, hemangioma, lymphangioma, carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyosarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, or Wilms tumor.
2.13 Any preceding Method, wherein the tumor is a brain tumor.
2.14 Method 2, wherein the gene product is a caspase gene or variant thereof selected from the group consisting of: caspase 1, caspase-3, caspase-4, caspase-5, caspase-11, and caspase-12.
2.15 Method 2.14, wherein the caspase gene or variant thereof are a pro-caspase gene (e.g, pro-caspase-1).
2.16 Methods 2.14-2.15, wherein the caspase gene or variant thereof is caspase-1.
2.17 Methods 2.14-2.16, wherein the caspase gene or variant thereof is a human, mouse, or rat caspase (e.g., human caspase-1, human pro-caspase 1).
2.18 Methods 2.14-2.17, wherein the gene product is delivered by viral vector.
2.19 Methods 2.18, wherein the viral vector comprises a heterologous gene/genes.
2.20 Method 2.19, wherein the heterologous gene/genes are selected from: genes not found in the viral genome; any allelic variant of a wild-type gene; and mutant gene(s).
2.21 Method 2.20, wherein Heterologous genes are preferably operably linked to a control sequence permitting expression of said heterologous gene in a cell in vivo.
2.22 Methods 2.18-2.21, wherein the viral vector can deliver a heterologous gene/genes to a cell in vivo where it will be expressed.
2.23 Methods 2.18-2.22, wherein the genes encode proteins which are cytostatic, cytotoxic, pro-drug activating, or which may be capable of stimulating/enhancing an immune response.
2.24 Methods 2.18-2.23, wherein the heterologous gene/genes are delivered by a single virus.
2.25 Methods 2.14-2.24, wherein the gene product comprises a viral vector comprising a pro-caspase gene (e.g., human pro-caspase-1) or a variant thereof and a promoter.
2.26 Methods 2.14-2.25, wherein the gene product further comprises an additional gene (e.g., human Caspase-3, human Caspase-4, human Caspase-5, human Caspase-11, mouse Caspase-11, mouse Caspase-12, NLRP1, Nlrp3, NRLC4, NALP, AIM2, PYCARD-derived adaptor proteins CARD or ASC, IL-β and IL-18).
2.27 Methods 2.14-2.26, wherein the gene product encodes a human pro-caspase-1 or a human caspase-1.
2.28 Methods 2.14-2.27, wherein the gene product is delivered by viral vector and wherein the viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated Virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
2.29 Methods 2.14-2.28, wherein the gene product is delivered by viral vector and wherein the viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, Adendo-assoicated Virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
2.30 Any of the preceding Methods, wherein the first sample and the second sample are taken from a patient to be treated for a cancer or tumor.

In various embodiments, the present disclosure also provides for a composition (Composition 2) comprising a compound or gene product identified in Method 2, et seq. for the treatment or amelioration of a cancer or tumor.

For example, Composition 2 may further encompass:
2.1 Composition 2, wherein the identified compound or gene product induces pyroptosis in a cancer or tumor cell.
2.2 Any preceding Composition, wherein the identified compound or gene product activates and causes release of a caspase, a pro-caspase, IL-β or IL-18, or combinations thereof.
2.3 Any preceding Composition, wherein the identified compound or gene product activates and causes release of caspase-1, caspase-3, caspase-4, caspase-5, caspase-11, caspase-12, IL-β or IL-18, or combinations thereof.
2.4 Any preceding Composition, wherein the identified compound or gene product activates and causes release of caspase-1, caspase-3, caspase-4, caspase-5, caspase-11, caspase-12 or combinations thereof.
2.5 Any preceding Composition, wherein the identified compound or gene product activates and causes release of a pro-caspase (e.g, pro-caspase-1).
2.6 Any preceding Composition, wherein the identified compound or gene product activates and causes release of caspase-1.
2.7 Any preceding Composition, wherein the identified compound or gene product causes release of pro-caspase-1.
2.8 Any preceding Composition, wherein the identified compound or gene product is administered in combination with an anticancer drug.
2.9 Any preceding Method, wherein the compound is useful in treating tumors.
2.10 Any preceding Method, wherein the tumor is selected from one or more of acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), schwannoma, adenomas (e.g., basophilic adenoma, eosinophilic adenoma, chromophobe adenoma, parathyroid adenoma, islet adenoma, fibroadenoma), fibroids (fibrous histiocytoma), fibromas, hemangiomas, lipomas (e.g., angiolipoma, myelolipoma, fibrolipoma, spindle cell lipoma, hibernoma, atypical lipoma), myxoma, osteoma, preleukemias, rhadomyoma, papilloma, seborrheic keratosis, skin adnexal tumors, hepatic adenomas, renal tubular adenoma, bile duct adenoma, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, nevus, actinic keratosis, cervical dysplasia, metaplasia (e.g., metaplasia of the lung), leukoplakia, hemangioma, lymphangioma, carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyosarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, or Wilms tumor.
2.11 Any of the preceding compositions, wherein the tumor is a brain tumor.
2.12 Any of the preceding Compositions, further comprising comprises a tumor cell promoter or tumor cell lineage promoter.
2.13 Any of the preceding Compositions, further comprising a ubiquitous promoter (e.g., CMV, CBA/CAG, and EF1a promoters).
2.14 Any of the preceding compositions, further comprising a tumor cell promoter or tumor cell lineage promoter that is upstream of the caspase gene or variant thereof.
2.15 Any of the preceding compositions, further comprising a promoter that is a human, mouse, or rat promoter.
2.16 Any of the preceding compositions, further comprising a replicative oncolytic viral vector or non-replicating viral vector comprises a heterologous gene/genes.
2.17 Composition 2.16, wherein the heterologous gene/genes are selected from: genes not found in the viral genome; any allelic variant of a wild-type gene; and mutant gene(s).
2.18 Composition 2.16-2.17, wherein Heterologous genes are preferably operably linked to a control sequence permitting expression of said heterologous gene in a cell in vivo.
2.19 Composition 2.16-2.18, wherein the replicative oncolytic viral or non-replicative vectors of the disclosure can deliver a heterologous gene/genes to a cell in vivo where it will be expressed.
2.20 Any of the preceding compositions, wherein the gene products typically encode proteins capable of enhancing the tumor destroying properties of the virus.
2.21 Any of the preceding compositions, wherein the gene products encode proteins which are cytostatic, cytotoxic, pro-drug activating, or which may be capable of stimulating/enhancing an anti-tumor immune response.
2.22 Composition 2.16-2.19, wherein the heterologous gene/genes are delivered by a single virus.
2.23 Any of the preceding compositions, wherein the composition can be administered oral, subcutaneous, intravenously, intratumorally, intraperitoneally or intravesicularly (kidneys).
2.24 Any of the preceding compositions, wherein the composition comprises an oncolytic viral vector or non-replicative viral vector comprising a pro-caspase gene (e.g., human pro-caspase-1) or a variant thereof and a tumor cell specific promoter or tumor cell lineage promoter.
2.25 Any of the preceding compositions, wherein the composition comprises a supernatant conditioned with the transfected cell.
2.26 Any of the preceding compositions, wherein the composition comprises an extracellular vesicle isolated from the transfected cell.
2.27 Any of the preceding compositions, wherein the composition comprises a pharmaceutically acceptable carrier.
2.28 Any of the preceding compositions, wherein the composition further comprises a chemotherapeutic agent.
2.29 Any of the preceding compositions, wherein the composition further comprises an additional gene (e.g., human Caspase-3, human Caspase-4, human Caspase-5, human Caspase-11, mouse Caspase-11, mouse Caspase-12, NLRP1, Nlrp3, NRLC4, NALP, AIM2, PYCARD-derived adaptor proteins CARD or ASC, IL-β and IL-18).
2.30 Any of the preceding compositions, wherein the virus is a replicative virus or non-replicative virus.
2.31 Any of the preceding compositions, wherein the oncolytic virus or non-replicative virus comprises a gene encoding a pro-caspase or a variant thereof (e.g., pro-caspase-1) and a tumor cell promoter or tumor cell lineage promoter.
2.32 Any of the preceding compositions, comprising a replicative oncolytic virus or non-replicative virus having a gene encoding caspase-1 or variant thereof and a tumor cell promoter or tumor cell lineage promoter (e.g., wherein the caspase-1 and the promoter are operably linked).
2.33 Composition 2.32, wherein the gene encodes a human pro-caspase-1 or a human caspase-1.
2.34 Any of the preceding compositions, comprising a replicative oncolytic virus or non-replicative virus that further comprises a suicide gene.
2.35 Composition 2.34, wherein the replicative oncolytic virus comprises a thymidine kinase suicide gene.
2.36 Any of the preceding compositions, wherein the composition comprises a supernatant obtained from a culture of a cell transfected with a replicative oncolytic vector or non-replicative vector comprising a caspase-1 gene or a variant thereof (e.g., human caspase-1 or human pro-caspase-1) and a tumor cell specific promoter or tumor cell lineage promoter.
2.37 Any of the preceding compositions, wherein the cell is a tumor cell (e.g., the transfected cell is cultured for about 12-24, 24-48, or 48-73 hours).
2.38 The composition of 2.36, wherein the supernatant comprises extracellular vesicles released from the transfected cell.
2.39 Any of the preceding compositions, wherein an extracellular vesicle is isolated from a cell transfected with a replicative oncolytic virus or non-replicative virus comprising a caspase-1 gene (e.g., human pro-caspase-1 or human caspase-1) or a variant thereof and a promoter.
2.40 The composition of 2.39, wherein the promoter can be a tumor cell specific promoter, a tumor cell lineage promoter or a ubiquitous promoter (e.g., CMV, CBA/CAG, and EF1 a promoter.
2.41 Any of compositions 2.16-2.40, wherein the replicative oncolytic vector or non-replicative vector further comprises a polyadenylation signal.
2.42 Composition 2.41, wherein the polyadenylation signal is downstream of a caspase-1 gene (e.g., human caspase-1 or pro-caspase-1) or the variant thereof.
2.43 Composition 2.42, wherein the polyadenylation signal comprises a bovine growth hormone polyadenylation signal, a SV40 polyadenylation signal or a rabbit beta-globin polyadenylation signal.
2.44 Any of Compositions 2.41-2.43, wherein the caspase gene (e.g., human caspase-1 or human pro-caspase-1) or the variant thereof and the polyadenylation signal are operably linked.
2.45 Any of the preceding compositions, comprising an oncolytic vector or non-replicative vector further having a first oncolytic viral vector or non-replicative viral vector inverted terminal repeat (ITR) located upstream of a tumor cell specific promoter or a tumor cell lineage promoter and a second oncolytic viral vector or non-replicative viral vector ITR located downstream of the polyadenylation signal.
2.46 The composition of 2.45, wherein the first or second replicative oncolytic viral vector or non-replicative viral vector inverted terminal repeat can include a deletion of the terminal resolution site.
2.47 Composition of 2.45 or 2.46, wherein the first and second ITRs, the tumor cell specific promoter or tumor cell lineage promoter, the caspase-1 gene or the variant thereof (e.g., human pro-caspase-1 or human caspase-1), and the polyadenylation signal are operably linked to constitute a functional replicative oncolytic vector or non-replicative vector capable of delivering caspase-1 (e.g., human pro-caspase-1 or human caspase-1) or a variant thereof to tumor cells or cancer.
2.48 Any of the preceding compositions, comprising an oncolytic vector or non-replicative vector further in a pharmaceutically acceptable carrier.
2.49 Any of the preceding compositions, wherein the composition further comprises a chemotherapeutic agent.
2.50 Composition 2.49, wherein the chemotherapeutic agent is selected from the group consisting of cyclophosphamide, vincristine, imidazole carboxamide, cisplatin, carboplatin, etoposide, gemcitabine, docetaxel, trabectedin, anthracycline, doxorubicin, ifosfamide, cycloxygenase inhibitors, non-steroidal anti-inflammatory agents and combinations thereof.
2.51 Any of the preceding compositions, wherein the composition is formulated for directed injection into a tumor.
2.52 Any of the preceding compositions, wherein the composition is encapsulated, tableted or prepared in an emulsion or syrup for oral administration.
2.53 Any of the preceding compositions, wherein the composition is formulated in an acceptable solid or liquid carrier (e.g., liquid carriers include syrup, peanut oil, olive oil, glycerin, saline, alcohols and water, and solid carriers can include, e.g., starch, lactose, calcium sulfate, dihydrate, terra alba, magnesium stearate or stearic acid, talc, pectin, acacia, agar or gelatin).
2.54 Any of the preceding compositions, wherein the carrier may comprise a sustained release material such as glyceryl monostearate or glyceryl distearate, alone or with a wax.
2.55 Any of the preceding compositions, wherein the replicative oncolytic viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
2.56 Any of the preceding pharmaceutical compositions, wherein the pharmaceutical composition comprises a replicative oncolytic viral vector or non-replicative viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
2.57 Any of the preceding Methods, wherein the first sample and the second sample are taken from a patient to be treated for a cancer or tumor.

The compounds or gene products identified via Method 2, et seq., may be used in pharmaceutical compositions to treat various types of cancers and tumors.

In some embodiments, the pharmaceutical compositions are administered in combination with one or more anticancer drugs, for example, drugs known to have an effect in treating or eliminating various types of cancers and/or tumors. Non-limiting examples of anticancer drugs are Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia, Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Ipilimumab, Tres sa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Maribo (Vincristine Sulfate Liposome), Matulane (Procarbazine Hydrochloride), Mechlorethamine Hqydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspagase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate).

In some embodiments, the methods and compositions may be used in the treatment of one or more types of tumors. For example, the present methods and compositions may be used in the treatment of benign tumors. Non-limiting examples of benign tumors includes adenomas (e.g., basophilic adenoma, eosinophilic adenoma, chromophobe adenoma, parathyroid adenoma, islet adenoma, fibroadenoma, renal tubular adenoma, bile duct adenoma, hepatic adenomas), fibroids (fibrous histiocytoma), fibromas, hemangiomas, lipomas (e.g., angiolipoma, myelolipoma, fibrolipoma, spindle cell lipoma, hibernoma, atypical lipoma), myxomas, osteomas, preleukemias, rhadomyomas, papillomas, seborrheic keratosis, skin adnexal tumors, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, and nevus.

In some embodiments, the present methods and compositions may be used in the treatment of pre-malignant tumors. Non-limiting examples of pre-malignant tumors includes actinic keratosis, cervical dysplasia, metaplasia (e.g., metaplasia of the lung), leukoplakia, hemangioma, and lymphangioma.

In some embodiments, the present methods and compositions may be used in the treatment of malignant tumors. Non-limiting examples of malignant tumors includes carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyosarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, and Wilms tumor.

In some embodiments, the present methods and compositions may be used in the treatment of brain tumors. Non-limiting examples of brain tumors includes acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), and schwannoma.

In a third aspect, the disclosure is directed to a method (Method 3) for identifying candidate compounds useful for inducing pyroptosis in a subject suffering from a bacterial, fungal or viral infection, the method comprising the step of:
a. providing a first sample and a second sample in which pyroptosis is inhibited, each sample containing equivalent amounts of a macrophage or dendritic cell;
b. contacting the first sample with one of the candidate compounds; and
c. measuring and comparing the amounts of inflammatory biomarkers in the first sample with the second sample, wherein an increased amount of the inflammatory biomarker in comparison with the second sample indicates that the candidate compound can be useful to treat or ameliorate bacterial, fungal or viral infections characterized by interrupted pyroptosis.
3.1 Method 3, further comprising the steps of:
   d. providing a third sample and a fourth sample in which pyroptosis is uninhibited, said third and fourth samples containing equivalent amounts of a macrophage or dendritic cell;
   e. contacting the third sample with one of the candidate compounds; and
   f. measuring and comparing the amounts of inflammatory biomarkers in the first sample with the third sample and/or fourth sample, wherein an increased amount of the inflammatory biomarker in comparison with the second sample indicates that the candidate compound can be useful to treat or ameliorate bacterial or viral infections characterized by interrupted pyroptosis.
3.2 Method 3, wherein the inflammatory biomarker is an inflammatory cytokine or a caspase enzyme.
3.3 Any preceding Method, wherein the inflammatory biomarker is a caspase, a pro-caspase, IL-β or IL-18, or combinations thereof.
3.4 Any preceding Method, wherein the inflammatory biomarker is caspase-1, caspase-3, caspase-4, caspase-5, caspase-11, caspase-12, IL-β or IL-18, or combinations thereof.
3.5 Any preceding Method, wherein the inflammatory biomarker is caspase-1, caspase-3, caspase-4, caspase-5, caspase-11, caspase-12 or combinations thereof.
3.6 Method 3 or 3.1-3.3, wherein the inflammatory biomarker is a pro-caspase (e.g, pro-caspase-1).
3.7 Any preceding Method, wherein the inflammatory biomarker is human caspase-1.
3.8 Any preceding Method, wherein the inflammatory biomarker is human pro-caspase-1.
3.9 Any preceding Method, wherein the first and second samples are infected with the same bacterial or viral infection.
3.10 Any preceding Method, wherein the first and second samples are infected with the same bacterial infection.
3.11 Any preceding Method, wherein the bacterial infection is a Salmonella, Shigella, Francisella, Listeria, Mycobacterium species or strain (e.g., Mycobacterium tuberculosis), Acinetobacter baumannii, Pseudomonas aeruginosa, Enterobacteriaceae, Enterococcus faecium, Staphylococcus aureus, Helicobacter pylori, Campylobacter, Neisseria gonorrhoeae, Streptococcus pneumoniae, Haemophilus influenzae infection.
3.12 Methods 3, 3.1-3.8, wherein the bacterial infection is a Mycobacterium tuberculosis infection.
3.13 Methods 3, 3.1-3.8 or 3.11-3.11, wherein the first and second samples originate from a single source.
3.14 Any preceding Method, wherein the macrophage or dendritic cell is an M1 or M2 macrophage.
3.15 Any preceding Method, wherein the macrophage or dendritic cell is an M1 macrophage.
3.16 Any preceding Method, wherein a successful candidate compound results in effective treatment against the infecting bacteria, fungus or virus.
3.17 Any preceding Method, wherein a successful candidate is marked by a 50% reduction in bacterial or viral viability, a 60% reduction in bacterial, fungal or viral viability, a 70% reduction in bacterial or viral viability, an 80% reduction in bacterial or viral viability, a 90% reduction in bacterial or viral viability, a 95% reduction in bacterial or viral viability, or greater.
3.18 Any preceding Method, wherein the gene product is a caspase gene or variant thereof selected from the group consisting of: caspase 1, caspase-3, caspase-4, caspase-5, caspase-11, and caspase-12.
3.19 Method 3.18, wherein the caspase gene or variant thereof are a pro-caspase gene (e.g, pro-caspase-1).
3.20 Methods 3.18-3.19, wherein the caspase gene or variant thereof is caspase-1.
3.21 Methods 3.17-3.19, wherein the caspase gene or variant thereof is a human, mouse, or rat caspase (e.g., human caspase-1, human pro-caspase 1)
3.22 Methods 3.18-3.21, wherein the gene product is delivered by viral vector.
3.23 Methods 3.18-3.22, wherein the viral vector comprises a heterologous gene/genes.
3.24 Method 3.23, wherein the heterologous gene/genes are selected from: genes not found in the viral genome; any allelic variant of a wild-type gene; and mutant gene(s).
3.25 Method 3.24, wherein Heterologous genes are preferably operably linked to a control sequence permitting expression of said heterologous gene in a cell in vivo.
3.26 Methods 3.22-3.25, wherein the viral vector can deliver a heterologous gene/genes to a cell in vivo where it will be expressed.
3.27 Methods 3.18-3.26, wherein the genes encode proteins which are cytotoxic, pro-drug activating, or which may be capable of stimulating/enhancing an immune response.
3.28 Methods 3.18-3.27, wherein the heterologous gene/genes are delivered by a single virus.
3.29 Methods 3.18-3.28, wherein the gene product comprises a viral vector comprising a pro-caspase gene (e.g., human pro-caspase-1) or a variant thereof and a promoter.
3.30 Methods 3.18-3.29, wherein the gene product further comprises an additional gene (e.g., human Caspase-3, human Caspase-4, human Caspase-5, human Caspase-11, mouse Caspase-11, mouse Caspase-12, NLRP1, Nlrp3, NRLC4, NALP, AIM2, PYCARD-derived adaptor proteins CARD or ASC, IL-1B and IL-18).
3.31 Methods 3.18-3.30, wherein the gene product encodes a human pro-caspase-1 or a human caspase-1.
3.32 Methods 3.18-3.31, wherein the gene product is delivered by viral vector and wherein the viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
3.33 Methods 3.18-3.32, wherein the gene product is delivered by viral vector and wherein the viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
3.34 Any of the preceding Methods, wherein the first sample and the second sample are taken from a patient to be treated for a bacterial, fungal or viral infection.

In a fourth aspect, the disclosure is directed to a method (Method 4) for identifying candidate compounds useful for inducing pyroptosis in a subject having cancer or a tumor, the method comprising the step of:
a. providing a first sample and a second sample, e.g., a cancerous cell or tissue sample;
b. contacting the first sample with one of the candidate compounds; and
c. measuring and comparing the amounts of inflammatory biomarkers in the first sample with the second sample, wherein an increased amount of the inflammatory biomarker in comparison with the second sample indicates that the candidate compound can be useful to treat or ameliorate a cancer or tumor characterized by interrupted pyroptosis.
4.1 Method 4, wherein the inflammatory biomarker is an inflammatory cytokine or a caspase enzyme.
4.2 Method 4 or 4.1, wherein the inflammatory biomarker is a caspase, a pro-caspase, IL-β or IL-18, or combinations thereof.
4.3 Any preceding Method, wherein the inflammatory biomarker is caspase-1, caspase-3, caspase-4, caspase-5, caspase-11, caspase-12, IL-β or IL-18, or combinations thereof.
4.4 Any preceding Method, wherein the inflammatory biomarker is caspase-1, caspase-3, caspase-4, caspase-5, caspase-11, caspase-12 or combinations thereof.
4.5 Method 2 or 2.1-2.2, wherein the inflammatory biomarker is a pro-caspase (e.g, pro-caspase-1).
4.6 Any preceding Method, wherein the inflammatory biomarker is human caspase-1.
4.7 Any preceding Method, wherein the inflammatory biomarker is human pro-caspase-1.
4.8 Any preceding Method, wherein the samples is cancerous cell or tissue.
4.9 Any preceding Method, wherein the first and second sample originates from a single source.
4.10 Any preceding Method, wherein the compound is useful in treating tumors.
4.11 Any preceding Method, wherein the tumor is benign, pre-malignant or malignant.
4.12 Any preceding Method, wherein the tumor is selected from one or more of acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), schwannoma, adenomas (e.g., basophilic adenoma, eosinophilic adenoma, chromophobe adenoma, parathyroid adenoma, islet adenoma, fibroadenoma), fibroids (fibrous histiocytoma), fibromas, hemangiomas, lipomas (e.g., angiolipoma, myelolipoma, fibrolipoma, spindle cell lipoma, hibernoma, atypical lipoma), myxoma, osteoma, preleukemias, rhadomyoma, papilloma, seborrheic keratosis, skin adnexal tumors, hepatic adenomas, renal tubular adenoma, bile duct adenoma, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, nevus, actinic keratosis, cervical dysplasia, metaplasia (e.g., metaplasia of the lung), leukoplakia, hemangioma, lymphangioma, carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyo sarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, or Wilms tumor.
4.13 Any preceding Method, wherein the tumor is a brain tumor.
4.14 Method 4, wherein the gene product is a caspase gene or variant thereof selected from the group consisting of: caspase 1, caspase-3, caspase-4, caspase-5, caspase-11, and caspase-12.
4.15 Method 4.14, wherein the caspase gene or variant thereof are a pro-caspase gene (e.g, pro-caspase-1).
4.16 Methods 4.14-4.15, wherein the caspase gene or variant thereof is caspase-1.
4.17 Methods 4.14-4.16, wherein the caspase gene or variant thereof is a human, mouse, or rat caspase (e.g., human caspase-1, human pro-caspase 1).
4.18 Methods 4.14-4.17, wherein the gene product is delivered by viral vector.
4.19 Methods 4.18, wherein the viral vector comprises a heterologous gene/genes.
4.20 Method 4.19, wherein the heterologous gene/genes are selected from: genes not found in the viral genome; any allelic variant of a wild-type gene; and mutant gene(s).
4.21 Method 4.20, wherein Heterologous genes are preferably operably linked to a control sequence permitting expression of said heterologous gene in a cell in vivo.
4.22 Methods 4.18-4.21, wherein the viral vector can deliver a heterologous gene/genes to a cell in vivo where it will be expressed.
4.23 Methods 4.18-4.22, wherein the genes encode proteins which are cytostatic, cytotoxic, pro-drug activating, or which may be capable of stimulating/enhancing an immune response.
4.24 Methods 4.18-4.23, wherein the heterologous gene/genes are delivered by a single virus.
4.25 Methods 4.14-4.24, wherein the gene product comprises a viral vector comprising a pro-caspase gene (e.g., human pro-caspase-1) or a variant thereof and a promoter.
4.26 Methods 4.14-4.25, wherein the gene product further comprises an additional gene (e.g., human Caspase-3, human Caspase-4, human Caspase-5, human Caspase-11, mouse Caspase-11, mouse Caspase-12, NLRP1, Nlrp3, NRLC4, NALP, AIM2, PYCARD-derived adaptor proteins CARD or ASC, IL-1□ and IL-18).
4.27 Methods 4.14-4.26, wherein the gene product encodes a human pro-caspase-1 or a human caspase-1.
4.28 Methods 4.14-4.27, wherein the gene product is delivered by viral vector and wherein the viral vector is selected from the group consisting of a: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated Virus, Newcastle Disease Virus (NDV), a polio virus, Moloney leukemia virus, a Mumps Virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
4.29 Methods 4.14-4.28, wherein the gene product is delivered by viral vector and wherein the viral vector selected from the group consisting of retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, Adendo-assoicated Virus, Newcastle Disease Virus (NDV), Moloney leukemia virus, a Mumps Virus, a polio virus, parvoviruses, a Measles Virus, a Vesicular Stomatitis Virus, a Para-influenza Virus, an Influenza Virus, a Herpes Simplex Virus I, Oncorine (H101), Onyx-015, RIGVIR, a Coxsackie virus, T-VEC, a Seneca Valley Virus, and a Reovirus.
4.30 Any of the preceding Methods, wherein the first sample and the second sample are taken from a patient to be treated for a cancer or tumor.

The disclosure also provides a kit (e.g., Kit 1) for treating, inhibiting, preventing, reducing the severity of and/or reducing the progression of a disease-state in a subject.

In one aspect the kit can comprise any of the Compositions disclosed herein (e.g., any of Composition 1 et seq. or Composition 2 et seq.) as well as any of the methods disclosed herein (e.g., any of Method 1 et seq., Method 2 et seq., or Method 3 et seq.).

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

“Oncolytic virus”, “oncolytic vectors” and “oncolytic viral vectors” are used interchangeably throughout. These refer to therapeutics which are designed to rapidly and specifically grow in tumors with the primary objective of directly lysing cancer cells. These are “replicative” viruses or viral vectors. These viruses are “replicative” in that they express viral gene products, replicate to form progeny virions, and are active viruses. Non-replicative viruses or viral vectors are not designed to grow in tumors but are designed to deliver viral gene products capable of either directly lysing cancer cells and/or bringing in immune cells along with their subsequent activation to kill cancer cells. Replicative oncolytic viral vectors and non-replicative viral vectors include, but are not limited to, the following: retroviral vector, vaccinia vector, lentiviral vector, baculoviral vector, cytomegalovirus (CMV) vector, simian virus (SV40) vector, Sindbis vectors, semliki forest virus, phage vectors, adenoviral (adenovirus) vectors, adeno-associated virus, Newcastle Disease Virus (NDV), Moloney leukemia virus, a Mumps Virus, parvoviruses, a Measles Virus, a Vesicular Stomatitis Virus, a Para-influenza Virus, an Influenza Virus, a polio virus, a Herpes Simplex Virus I, Oncorine (H101), Onyx-015, a Coxsackie virus, RIGVIR, T-VEC, a Seneca Valley Virus, and a Reovirus.

“Caspase” as used herein, can refer to either “pro-caspase” or “caspase”. Caspases are synthesized as inactive zymogens (pro-caspases) that are only activated following an appropriate stimulus. This post-translational level of control allows rapid and tight regulation of the enzyme. Activation involves dimerization and often oligomerization of pro-caspases, followed by cleavage into a small subunit and large subunit. The large and small subunit associate with each other to form an active heterodimer caspase. The active enzyme often exists as a heterotetramer in the biological environment, where a pro-caspase dimer is cleaved together to form a heterotetramer.

“Promoter” as used herein refers to a region of DNA that initiates transcription of a particular gene. Promoters are located near the transcription start sites of genes, on the same strand and upstream on the DNA (towards the 5′ region of the sense strand). Promoters described here or either tumor cell specific promoters or tumor cell lineage promoters. Tumor cell lineage promoters are promoters not specific for tumor cells but are specific to the cells from which the tumor cell originates.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “carcinoma” refers to a cancer arising from epithelial cells. As used herein, the term “invasive” refers to the ability to infiltrate and destroy surrounding tissue. Melanoma is an invasive form of skin tumor.

The term “sample” or “biological sample” as used herein denotes a sample taken or isolated from a biological organism, e.g., a tumor sample, bacterium, fungus or virus from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy or a bacterium, fungus or virus taken from biofluid sample; serum; plasma; urine; saliva or tissue from an organ.

The term “functional” when used in conjunction with “derivative” or “variant” or “fragment” refers to a polypeptide which possess a biological activity that is substantially similar to a biological activity of the entity or molecule of which it is a derivative or variant or fragment thereof. By “substantially similar” in this context is meant that at least 25%, at least 35%, at least 50% of the relevant or desired biological activity is retained.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

As used herein, the word “toxic” means effects on cells (e.g., tumor cells) that result in cell death, reduced ability to divide, or cell population reduction.

As used herein, “variants” can include, but are not limited to, those that include conservative amino acid mutations, SNP variants, splicing variants, degenerate variants, and biologically active portions of a gene. A “degenerate variant” as used herein refers to a variant that has a mutated nucleotide sequence, but still encodes the same polypeptide due to the redundancy of the genetic code. In accordance with the present disclosure, the caspase gene (e.g., caspase-1) may be modified, for example, to facilitate identification and/or improve expression, so long as such modifications do not reduce caspase-1's function to unacceptable level. In various embodiments, a variant of the caspase gene (e.g., caspase-1) encodes a protein that has at least 10%, 20%, 30%, 40%, 50%, 60%, 70%, 80%, 90%, or 95% of the function of a wild-type caspase protein (e.g., caspase-1).

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. See, e.g., U.S. Pat. No. 5,399,346, the contents of which are incorporated herein by reference.

“Vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

“Recombinant virus” refers to a virus that has been genetically altered (e.g., by the addition or insertion of a heterologous nucleic acid construct into the particle).

The term “transfection” is used herein to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197, the contents of each of which are incorporated herein by reference. Such techniques can be used to introduce one or more exogenous DNA moieties, such as a plasmid vector and other nucleic acid molecules, into suitable host cells. The term refers to both stable and transient uptake of the genetic material.

The term “transduction” denotes the delivery of a DNA molecule to a recipient cell either in vivo or in vitro, via any method of gene delivery. For example, transduction could mean uptake of a virus, part of which is DNA.

The term “heterologous,” as it relates to nucleic acid sequences such as gene sequences and control sequences, denotes sequences that are not normally joined together and/or are not normally associated with a particular virus. Allelic variation or naturally occurring mutational events do not give rise to heterologous DNA, as used herein.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Homology” and “homologous” as used herein refer to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s), and size determination of the digested fragments. Two DNA or two polypeptide sequences are “substantially homologous” to each other when at least about 80%, preferably at least about 90%, and most preferably at least about 95% of the nucleotides or amino acids, respectively, match over a defined length of the molecules, as determined using the methods above.

“Isolated” as used herein when referring to a nucleotide sequence, vector, etc., refers to the fact that the indicated molecule is present in the substantial absence of other biological macromolecules of the same type. Thus, an “isolated nucleic acid molecule which encodes a particular polypeptide” refers to a nucleic acid molecule that is substantially free of other nucleic acid molecules that do not encode the subject polypeptide. Likewise, an “isolated vector” refers to a vector that is substantially free of other vectors that differ from the subject vector. However, the subject molecule or vector may include some additional bases or moieties that do not deleteriously affect the basic characteristics of the composition.

“Purified” as used herein when referring to a vector, refers to a quantity of the indicated vector that is present in the substantial absence of other biological macromolecules. Thus, a “purified vector” refers to a composition that includes at least 80% subject vector, preferably at least 90% subject vector, most preferably at least 95% subject vector with respect to other components of the composition.

“Extracellular vesicles” (EVs, e.g., exosomes, microvesicles, microparticles, circulating microvesicles, shedding microvesicles, nanovesicles, nanoparticles, apoptotic bodies, and membrane vesicles) are fragments of plasma membrane ranging from for example, 20 nm to 10 μm, shed from almost all cell types. Microvesicles play a role in intercellular communication and can transport mRNA, miRNA, and proteins between cells. As will be apparent to a person of skill in the art, there are various EV isolation and purification protocols based on filtration, differential centrifugation, ultracentrifugation, flotation of vesicles in gradients (sucrose, OptiPrep™), and immunoaffinity capture utilizing antibodies against membrane proteins. Exemplary information for isolating extracellular vesicles may be found in Simpson R J, Mathivanan S (2012) Extracellular Microvesicles: The Need for Internationally Recognised Nomenclature and Stringent Purification Criteria. J Proteomics Bioinform 5: ii-ii; van der Pol et al., Classification, functions, and clinical relevance of extracellular vesicles, Pharmacol Rev. 2012 July; 64(3):676-705; Raposo and Stoorvogel, Extracellular vesicles: exosomes, microvesicles, and friends, J. Cell Biol. 2013 Feb. 18; 200(4): 373-83; and Witwer et al., Standardization of sample collection, isolation and analysis methods in extracellular vesicle research, J Extracell Vesicles. 2013 May 27; 2, which are incorporated herein by reference in their entirety. Also, see Sarkar et al., 2009, Taylor and Gercel-Taylor, 2008, and Balaj et al., 2011, the contents of each of which are incorporated herein by reference in their entireties.

“Schwannoma tumors” are composed of Schwann-lineage cells, and can refer to tumors which form along the peripheral, spinal and cranial nerves. These tumors can cause pain, sensory/motor dysfunction, and death through compression of peripheral nerves, the spinal cord, and/or the brain stem.

“Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The terms “bacterial infection”, “fungal infection” and “viral infection” as used herein refers to presence of bacteria, fungus or a virus, in or on a subject, which, if its growth or replication were inhibited, would result in a benefit to the subject. As such, the term “infection” in addition to referring to the presence of bacteria, fungus and virus also refers to normal flora that are not desirable. The terms “infection” or “bacterial infection” also include infections caused by gram-positive and gram-negative bacteria.

The term “antibacterial agent”, “anti-fungal agent” or “anti-viral agent” as used herein refers to any substance, compound or a combination of substances or a combination compounds capable of: (i) inhibiting, reducing or preventing growth of bacteria, fungus or virus; (ii) inhibiting or reducing ability of a bacteria, fungus or virus to produce infection in a subject; or (iii) inhibiting or reducing ability of bacteria, fungus or virus to multiply or remain infective in the environment. The term “antibacterial agent”, “anti-fungal agent” or “anti-viral agent” also refers to compounds capable of decreasing infectivity or virulence of bacteria, fungus or virus.

The pharmaceutical preparations of the present disclosure may be made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the disclosure (e.g., any of Composition 1 et seq. or Composition 2 et seq.) may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Before administration to patients, formulants may be added to the compositions. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water-soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxyethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some aspects, polymers as formulants include polyvinylpyrrolidone (PVP) with an average molecular weight between 2,000 and 3,000, or polyethylene glycol (PEG) with an average molecular weight between 3,000 and 5,000.

In some aspects, a buffer is used in the composition to minimize pH changes in the solution before lyophilization or after reconstitution. A number of physiological buffers including, but not limited to: citrate, phosphate, succinate, and glutamate buffers or mixtures thereof. In some aspects, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

In various aspects, the pharmaceutical composition is administrated to the subject before, during, or after the subject develops the condition. In various embodiments, the composition is administrated to the subject oral, intranervously, intracranially, intratumorally, intramuscularly, intravenously, intradermally, or subcutaneously, or a combination thereof

In various aspects, the therapeutically effective amount of the composition comprises about 1×10⁹-1×10¹⁴, 1×10⁹-1×10¹², 1×10¹⁰-1×10¹², 1×10¹²-1×10¹⁴, 1×10¹²-1×10¹⁵ or 1×10¹⁵-1×10¹⁸genome copies (gc) of the r vector per kg of body weight of the subject (e.g., any of Compositions 1.0 et seq).

In various aspects, the composition (e.g., any of Composition 1.0 et seq) is administrated to the subject 1-3 times per day or 1-7 times per week. In various embodiments, the composition is administrated to the subject for 1-5 days, 1-5 weeks, 1-5 months, 1-5 years or throughout the life time of the subject as deemed necessary and would be apparent to a person of skill in the art.

In various aspects, the method further comprises treating the subject with surgery, radiation therapy, or chemotherapy, or a combination thereof. The surgery, radiation therapy, or chemotherapy, or a combination thereof may be conducted before, during or after administering a therapeutically effective amount of the composition to the subject.

“Surgeries” as used herein, may include, but are not limited to, limb sparing surgery, amputation, and plastic surgery. Examples of radiation therapies include but are not limited to stereotactic radiotherapy. In this procedure, a large, precise, external dose of radiation is delivered to the area where, e.g., the schwannoma is located. It can be given once or over the course of several treatment sessions. Examples of chemotherapeutic agents used for chemotherapy include but are not limited to any one or more of cyclophosphamide, vincristine, imidazole carboxamide, cisplatin, carboplatin, etoposide, gemcitabine, docetaxel, trabectedin, anthracycline, doxorubicin, ifosfamide, cycloxygenase inhibitors, non-steroidal anti-inflammatory agents or combinations thereof.

1. A method for identifying candidate compounds or gene products useful in treating or ameliorating bacterial, fungal or viral infections comprising the steps of:
a. providing a first sample and a second sample, e.g., a bacterial, fungal or viral culture or cell or tissue sample infected with a bacteria, fungus or virus, containing equivalent amounts of a macrophage or dendritic cell;
b. contacting the first sample with one of the candidate compounds or gene products;
c. and determining whether the amounts of an inflammatory biomarker in the first sample has changed, wherein an increased amount of the inflammatory biomarker in comparison with the second sample indicates that the candidate compound can be useful to treat or ameliorate bacterial, fungal or viral infections characterized by interrupted pyroptosis.

2. A method according to claim 1, further comprising the steps of
d. providing a third sample and a fourth sample e.g., a bacterial culture or cell or tissue sample, both free of infection from the bacteria, fungus or virus, said third and fourth samples containing equivalent amounts of a macrophage or dendritic cell;
e. contacting the third sample with one of the candidate compounds or gene products; and
f. measuring and comparing the amounts of inflammatory biomarkers in the first sample with the third sample and/or fourth sample, wherein an increased amount of the inflammatory biomarker in comparison with the second sample indicates that the candidate compound can be useful to treat or ameliorate bacterial or viral infections characterized by interrupted pyroptosis.

3. A method according to claim 1, wherein the inflammatory biomarker is an inflammatory cytokine or a caspase enzyme.

4. A method according to claim 1, wherein the inflammatory biomarker is a caspase, a pro-caspase, IL-β or IL-18, or combinations thereof.

5. A method according to claim 1, wherein the inflammatory biomarker is pro-caspase-1, caspase-1, caspase-3, caspase-4, caspase-5, caspase-11, caspase-12, IL-β or IL-18, or combinations thereof.

6. A method according to claim 1, wherein the bacterial infection is a Salmonella, Shigella, Francisella, Listeria, Mycobacterium species or strain (i.e., Mycobacterium tuberculosis), Acinetobacter baumannii, Pseudomonas aeruginosa, Enterobacteriaceae, Enterococcus faecium, Staphylococcus aureus, Helicobacter pylori, Campylobacter, Neisseria gonorrhoeae, Streptococcus pneumoniae, Haemophilus influenzae infection.

7. A method according to claim 1, wherein a successful candidate is marked by a 50% reduction in bacterial, fungal or viral viability, a 60% reduction in bacterial or viral viability, a 70% reduction in bacterial or viral viability, an 80% reduction in bacterial or viral viability, a 90% reduction in bacterial or viral viability, a 95% reduction in bacterial or viral viability, or greater.

8. A method for identifying candidate compounds useful in treating or ameliorating a cancer or tumor comprising the steps of:
a. providing a sample, e.g., a cancerous cell or tissue sample;
b. contacting the sample with one of the candidate compounds;
c. and determining whether the amounts of an inflammatory biomarker in the sample has changed, wherein an increased amount of the inflammatory biomarker in comparison with the sample lacking candidate compound can be useful to treat or ameliorate a tumor or cancer characterized by interrupted pyroptosis.

9. A method according to claim 8, wherein the inflammatory biomarker is an inflammatory cytokine or a caspase enzyme.

10. A method according to claim 8, wherein the inflammatory biomarker is a caspase, a pro-caspase, IL-β or IL-18, or combinations thereof.

11. A method according to claim 8, wherein the inflammatory biomarker is pro-caspase-1, caspase-1, caspase-3, caspase-4, caspase-5, caspase-11, caspase-12, IL-β or IL-18, or combinations thereof.

12. A method according to claim 8, wherein the tumor is selected from one or more of acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), schwannoma, adenomas (e.g., basophilic adenoma, eosinophilic adenoma, chromophobe adenoma, parathyroid adenoma, islet adenoma, fibroadenoma), fibroids (fibrous histiocytoma), fibromas, hemangiomas, lipomas (e.g., angiolipoma, myelolipoma, fibrolipoma, spindle cell lipoma, hibernoma, atypical lipoma), myxoma, osteoma, preleukemias, rhadomyoma, papilloma, seborrheic keratosis, skin adnexal tumors, hepatic adenomas, renal tubular adenoma, bile duct adenoma, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, nevus, actinic keratosis, cervical dysplasia, metaplasia (e.g., metaplasia of the lung), leukoplakia, hemangioma, lymphangioma, carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyosarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, or Wilms tumor.

13. A method for identifying candidate compounds useful for inducing pyroptosis in a subject suffering from a bacterial, fungal or viral infection, or a cancer or tumor, the method comprising the step of:
a. providing a first sample and a second sample in which pyroptosis is inhibited, containing equivalent amounts of a macrophage or dendritic cell;
b. contacting the first sample with one of the candidate compounds;
c. and determining whether the amounts of an inflammatory biomarker in the first sample has changed, wherein an increased amount of the inflammatory biomarker in comparison with the second sample indicates that the candidate compound can be useful to treat or ameliorate bacterial, fungal or viral infections, or a cancer or tumor characterized by interrupted pyroptosis.

14. A method according to claim 13, further comprising the steps of:
d. providing a third sample and a fourth sample in which pyroptosis is uninhibited, said third and fourth samples containing equivalent amounts of a macrophage or dendritic cell;
e. contacting the third sample with one of the candidate compounds; and
f. measuring and comparing the amounts of inflammatory biomarkers in the first sample with the third sample and/or fourth sample, wherein an increased amount of the inflammatory biomarker in comparison with the second sample indicates that the candidate compound can be useful to treat or ameliorate bacterial or viral infections characterized by interrupted pyroptosis.

15. A method according to claim 13, wherein the inflammatory biomarker is an inflammatory cytokine or a caspase enzyme.

16. A method according to claim 13, wherein the inflammatory biomarker is a caspase, a pro-caspase, IL-β or IL-18, or combinations thereof.

17. A method according to claim 13, wherein the inflammatory biomarker is pro-caspase-1, caspase-1, caspase-3, caspase-4, caspase-5, caspase-11, caspase-12, IL-β or IL-18, or combinations thereof.

18. A method according to claim 13, wherein the tumor is selected from one or more of acoustic neuroma, astrocytoma, chordoma, CNS lymphoma, craniopharyngioma, gliomas (e.g., Brain stem glioma, ependymoma, mixed glioma, optic nerve glioma, subependymoma, medulloblastoma, meningioma, metastatic brain tumors, oligodendroglioma, pituitary tumors, primitive neuroectodermal (PNET), schwannoma, adenomas (e.g., basophilic adenoma, eosinophilic adenoma, chromophobe adenoma, parathyroid adenoma, islet adenoma, fibroadenoma), fibroids (fibrous histiocytoma), fibromas, hemangiomas, lipomas (e.g., angiolipoma, myelolipoma, fibrolipoma, spindle cell lipoma, hibernoma, atypical lipoma), myxoma, osteoma, preleukemias, rhadomyoma, papilloma, seborrheic keratosis, skin adnexal tumors, hepatic adenomas, renal tubular adenoma, bile duct adenoma, transitional cell papilloma, hydatidiform moles, ganglioneuroma, meningoma, neurilemmoma, neurofibroma, C cell hyperplasia, pheochromocytoma, insulinoma, gastrinoma, carcinoids, chemodectoma, paraganglioma, nevus, actinic keratosis, cervical dysplasia, metaplasia (e.g., metaplasia of the lung), leukoplakia, hemangioma, lymphangioma, carcinoma (e.g., squamous cell carcinoma, epidermoid carcinoma, adenocarcinoma, hepatoma, hepatocellular carcinoma, renal cell carcinoma, cholangiocarcinoma, transitional cell carcinoma, embryonal cell carcinoma, parathyroid carcinoma, medullary carcinoma of thyroid, bronchial carcinoid, oat cell carcinoma, islet cell carcinoma, malignant carcinoid,), sarcoma (e.g., fibrosarcoma, myxosarcoma, liposarcoma, chondrosarcoma, osteosarcoma, malignant fibrous histiocytoma, hemangiosarcoma, angiosarcoma, lymphangiosarcoma, leiomyosarcoma, rhabdomyosarcoma, neurofibrosarcoma), blastoma (e.g., medulloblastoma and glioblastoma, types of brain tumor, retinoblastoma, a tumor in the retina of the eye, osteoblastoma, bone tumors, neuroblastoma), germ cell tumor, mesothelioma, malignant skin adnexal tumors, hypernephroma, seminoma, glioma, malignant meningioma, malignant shwannoma, malignant pheochromocytoma, malignant paraganglioma, melanoma, mercell cell neoplasm, cystosarcoma phylloides, or Wilms tumor.

19. A method according to claim 13, wherein the bacterial infection is a Salmonella, Shigella, Francisella, Listeria, Mycobacterium species or strain (i.e., Mycobacterium tuberculosis), Acinetobacter baumannii, Pseudomonas aeruginosa, Enterobacteriaceae, Enterococcus faecium, Staphylococcus aureus, Helicobacter pylori, Campylobacter, Neisseria gonorrhoeae, Streptococcus pneumoniae, Haemophilus influenzae infection.

20. A method according to claim 13, wherein a successful candidate is marked by a 50% reduction in bacterial, fungal or viral viability, a 60% reduction in bacterial or viral viability, a 70% reduction in bacterial or viral viability, an 80% reduction in bacterial or viral viability, a 90% reduction in bacterial or viral viability, a 95% reduction in bacterial or viral viability, or greater.
